(19) United States
(12) Patent Application Publication
(10) Pub. No.: US 2025/0152402 A1
(43) Pub. Date: May 15, 2025

(54) CHARGING DOCK FOR OSTOMY LEAKAGE DETECTION SYSTEM

(71) Applicant: HOLLISTER INCORPORATED, Libertyville, IL (US)

(72) Inventors: JONAS P. CARLSSON, Chicago, IL (US); Lauren M. Lattanzi, Raleigh, NC (US); Anthony B. Smith, Durham, NC (US); Grace E. Rhinehart, Cary, NC (US); Elise Jaffe, Cary, NC (US)

(73) Assignee: HOLLISTER INCORPORATED, Libertyville, IL (US)

(21) Appl. No.: 18/840,151

(22) PCT Filed: Mar. 7, 2023
(86) PCT No.: PCT/US2023/063857

§ 371 Date: Aug. 21, 2024

Related U.S. Application Data
(60) Provisional application No. 63/321,855, filed on Mar. 21, 2022.
(60) Provisional application No. 63/407,925, filed on Sep. 19, 2022.

(51) Int. Cl. A61F 5/445

(52) U.S. Cl. CPC A61F 5/445

(57) ABSTRACT

A charging dock for charging a wearable device for an ostomy leakage detection system. The charging dock may include a housing, a slide-in mechanism, a contact pin, and a ramp. The housing may include a first and second wall. The slide-in mechanism may be defined by the first and second wall and configured to receive a wearable device. The first wall may include a first opening for accessing the wearable device. The first wall may include at least one peripheral edge that may be configured to extend over the wearable device and prevent the wearable device from opening while docked. The contact pin may be located within the slide-in mechanism and may be configured to charge the wearable device. The ramp may be located within the slide-in mechanism and configured to charge to couple with the wearable device to prevent rotation of the wearable device while docked.

### BACKGROUND

This disclosure is related to a leakage detection system. More particularly, the present disclosure pertains to a charging dock for a wearable device of the leakage detection system.

Known ostomy leakage detection systems can include subsystems which comprise a wearable device for alerting a user to the presence of ostomy effluent (or leakage) under their hydrocolloid barrier. Commonly, such wearable devices can include a rechargeable battery and can supply the electrical power needed to detect moisture and communicate with the user. Accordingly, the wearable device must be recharged to replenish power to facilitate continued use. Currently, there are charging docks for charging such wearable devices. However, securely docking and un-docking a wearable device designed for an ostomy leakage detection system may be difficult to some users. For example, the wearable device may have specific docking requirements and a user may only have one free hand for docking and un-docking the wearable device.

Accordingly, it is desirable to provide an easy-to-use charging dock for the wearable device.

### BRIEF SUMMARY

A charging dock for a wearable device of a leakage detection system is provided according to example embodiments.

In one aspect, the charging dock may include a housing, a slide-in mechanism, a contact pin, and a ramp. The housing may include a first and second wall. The slide-in mechanism may be defined by the first and second wall and configured to receive a wearable device. The first wall may include a first opening for accessing the wearable device. The first wall may include at least one peripheral edge that may be configured to extend over the wearable device and prevent the wearable device from opening while docked. The contact pin may be located within the slide-in mechanism and may be configured to charge the wearable device. The ramp may be located within the slide-in mechanism and configured to charge to couple with the wearable device to prevent rotation of the wearable device while docked.

In an embodiment, the contact pin may include springs.

In an embodiment, the second wall may include a second opening for accessing the wearable device. The second wall may include at least one peripheral edge that extends over the wearable device.

In an embodiment, the housing may include a top housing and a bottom housing. The top housing may include the first and second wall. In such embodiment, the bottom housing may include a base material configured to provide friction. The base material may include an elastomer.

In an embodiment, the bottom housing may include a weight plate configured to provide stability and prevent the charging dock from sliding on a flat surface.

In an embodiment, the housing may include the first opening allows for a light on the wearable device to be visible. The light may include a charging status.

In an embodiment, the first and second openings may be configured to allow the wearable device to be undocked with one hand.

In another aspect, charging dock may include a top housing, a bottom housing, a slide-in mechanism, and a base material. The bottom housing may be connected to the top housing. The bottom housing may include a weight plate configured to provide stability and prevent the charging dock from sliding on a surface. The slide-in mechanism may be defined in the top housing. The slide-in mechanism may include contact pins configured to charge a wearable device. The base material may be located on the bottom housing and configured to provide friction between the charging dock and a surface.

In an embodiment, the base material may include an elastomer.

In an embodiment, the top housing may include a first and second wall. The first wall may include a first opening for accessing the wearable device. The first wall may include at least one peripheral edge that may extend over the wearable device and may be configured to prevent the wearable device from opening while docked. In such an embodiment, the second wall may include a second opening for accessing the wearable device. The second wall may include at least one peripheral edge that extends over the wearable device.

In an embodiment, the first and second openings may be configured to allow the wearable device to be un-docked with one hand.

In an embodiment, the charging dock may further include a ramp located within the slide-in mechanism and may be configured to couple with the wearable device to prevent rotation of the wearable device while docked.

In yet another aspect, a method may include receiving a wearable device into a charging dock, providing tactile and auditory feedback as a contact pin passes a protrusion member and is decompressed, engaging the contact pin with a charging pad, and engaging an aligning opening with a ramp to secure the contact pin on the charging pad. The wearable device may include the aligning opening, the protrusion member, and the charging pad. The charging dock may include a housing, a slide-in mechanism defined by a first and second wall, the contact pin located within the slide-in mechanism and configured to charge the wearable device, and the ramp located within the slide-in mechanism. The housing may include the first and second wall. The contact pin may include a spring.

In an embodiment, the method may further include securing a latching mechanism between the first and second wall. The latching mechanism may be located on the outer edge of the wearable device and may not be opened within the charging dock.

In an aspect, a charging dock for charging a wearable device may comprise a magnetic docking mechanism configured to pull and secure the wearable device in a charging position. The charging dock may include a magnet and spring contact pins, and the wearable device may include a metal pin. The charging dock may be configured such that when the wearable device is magnetically pulled into the charging position, the spring contact pins are depressed by the wearable device and an electrical connection between the charging dock and the wearable device is established.

In another aspect, a charging dock for charging a wearable device may comprise a base, a hinged top, and a magnet-magnet or magnet-ferromagnetic metal connection configured to magnetically attach the hinged top to the base in a closed position. The base may be configured to receive a wearable device and include spring contact pins. The hinged top may be connected to the base via a hinge and include an opening. The charging dock may be configured such that the hinged top clamps over the wearable device when the wearable device is received in the base and the charging dock is closed and in a charging position. Atop portion of the wearable device may be visible through the opening when the charging dock is closed.

In yet another aspect, a charging dock for charging a wearable device may include a latch mechanism configured to secure the wearable device in a charging position. The latch mechanism may include first and second panel latches, each attached to a side of the charging dock. The latch mechanism may be configured such that when the panel latches are simultaneously squeezed at lower portions, upper portions of the panel latches hinge outwardly to receive the wearable device. When released, the panel latches may latch over the wearable device to secure the wearable device in the charging position.

In an aspect, a charging dock for charging a wearable device may include a base configured to receive a wearable device, an upper portion, and a press tab release mechanism. The press tab release mechanism may include a tab and an internal spring coil connecting the upper portion and the tab at a hinge. The charging dock may be configured such that the upper portion can be opened by pressing down the tab to receive the wearable device into the base or to remove the wearable device from the base. When the wearable device is received in the base and the tab is released, the upper portion may clamp over the wearable device to establish an electrical connection between the wearable device and the charging dock in a charging position.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is a schematic illustration of an ostomy system, according to an embodiment.

FIG. 1B is an illustration of an ostomy system attached to a user, according to an embodiment.

FIG. 2 is a front perspective view of a charging dock with a “slide-in” mechanism according to an embodiment.

FIG. 3 is a front perspective view of the charging dock of FIG. 2 and a wearable device aligned for docking.

FIG. 4 is a back elevation view of the charging dock of FIG. 2 and the wearable device aligned for docking.

FIG. 5 is a front perspective view of the charging dock of FIG. 2 and the wearable device fully inserted in the charging position.

FIG. 6 is a side elevation view of the charging dock of FIG. 2.

FIG. 7 is a top perspective view of the charging dock of FIG. 2.

FIG. 8 is a partial top view of the charging dock of FIG. 2.

FIG. 9 is a partial side view of the charging dock of FIG. 2.

FIG. 10 is a bottom perspective view of the charging dock of FIG. 2.

FIG. 11 is a bottom view of the charging dock of FIG. 2.

FIG. 12 is a cross-sectional side view of the charging dock of FIG. 2.

FIG. 13 is a bottom cross-sectional side view of the charging dock of FIG. 2.

FIG. 14 is an exploded perspective view of the charging dock of FIG. 2.

FIG. 15 is a perspective view of a charging dock and a wearable device aligned for docking according to an embodiment.

FIG. 16 is a cross-sectional view of the charging dock of FIG. 15 with the wearable device docked in a charging position.

FIG. 17 is a perspective view of a charging dock in an open position and a wearable device aligned for docking according to an embodiment.

FIG. 18 is a cross-sectional view of the charging dock of FIG. 17 with the wearable device docked in a closed and charging position.

FIG. 19 is a perspective view of a charging dock and a wearable device aligned for docking according to an embodiment.

FIG. 20 is a perspective view of the charging dock of FIG. 19 with the wearable device received in the charging dock for charging.

FIG. 21 is a perspective view of a charging dock including a press tab release mechanism in a closed position with a wearable device received therein according to an embodiment.

FIG. 22 is an enlarged partial sectional view of the press tab release mechanism of the charging dock of FIG. 21.

FIG. 23 is a perspective view of the charging dock of FIG. 21 in an open position with the wearable device aligned for docking.

FIG. 24 is an enlarged partial sectional view of the press tab release mechanism of the charging dock of FIG. 23.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,” “down,” front,” “back,” and the like are used for purposes of illustration and as such are not limiting. Depending on the context, the word “if” as used herein may be interpreted as “when” or “upon” or “in response to determining.”

The present disclosure provides a charging dock for an ostomy leakage detection system. The ostomy leakage detection system can be configured to detect ostomy effluent leakage under a skin barrier and to alert a user. The ostomy leakage detection system can provide multiple benefits to the user. For example, the system can allow the user to intervene and change a skin barrier and/or ostomy pouch system before a leak progresses which can cause embarrassment and inconvenience to the user. Further, the ostomy leakage detection system can assist in maintaining a user's skin health by detecting a leakage in its early stage to prevent prolonged skin exposure to ostomy effluent, which can lead to skin health complications. The ostomy leakage detection system can also support a user's emotional well-being by reducing anxiety associated with a risk of leakage. The ostomy leakage detection system may be applied to an ostomy barrier of a one-piece pouch system or a faceplate for a two-piece pouch system.

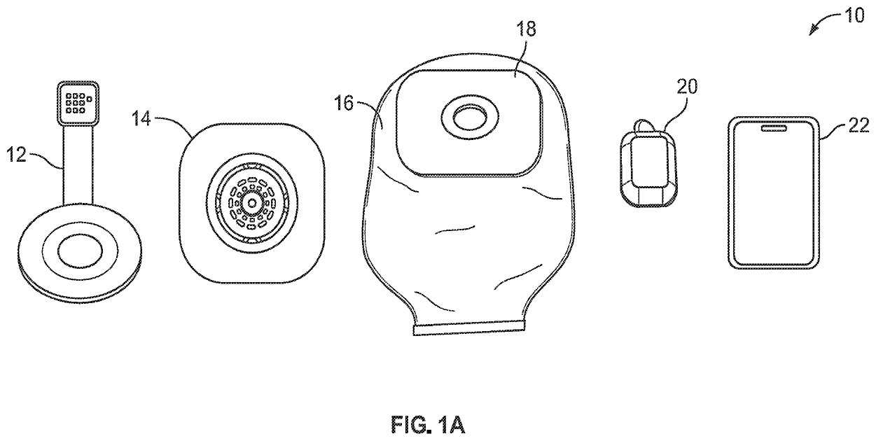

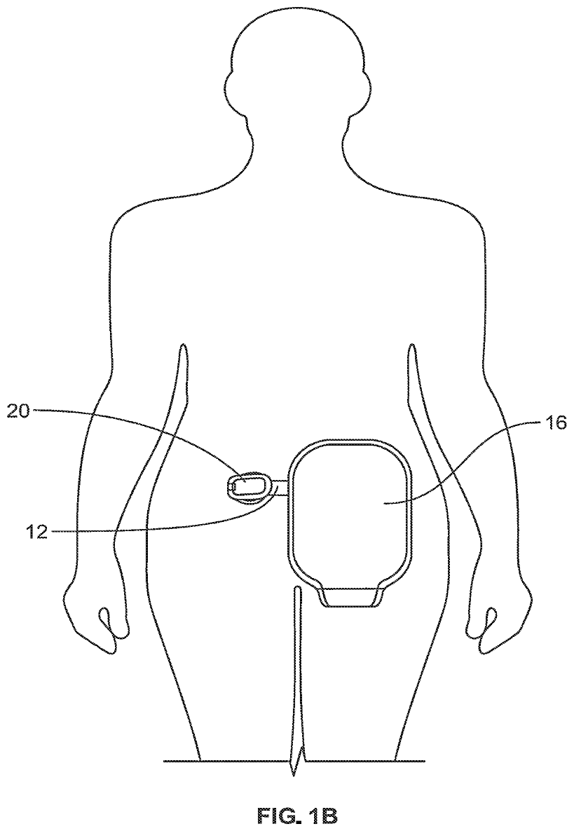

FIG. 1A illustrates an ostomy two-piece pouch system 10. According to example embodiments shown schematically in FIG. 1A, the ostomy system 10 can generally include a sensing accessory 12, an ostomy barrier appliance 14, an ostomy bag 16 with an ostomy barrier coupling member 18, a wearable subsystem 20, and a mobile device 22.

FIG. 1B illustrates the ostomy pouch system 10 attached to a user. According to example embodiments shown in FIG. 1B, a first end of the sensing accessory 12 can be attached to the user and surround a stoma. The sensing accessory 12 can be designed to detect moisture around the stoma. The ostomy barrier appliance 14 can be attached over the sensing accessory 12. The ostomy bag 16 can be connected to the ostomy barrier appliance 14 using the ostomy barrier coupling member 18. The wearable subsystem 20 can be attached to a second end of the sensing accessory 12.

According to example embodiments, the ostomy leakage detection system may comprise three subsystems—the sensing accessory 12, the wearable subsystem 20, and a mobile application on the mobile device 22. The sensing accessory 12 may be provided as an accessory for an ostomy pouch system. The sensing accessory may include sensors for detecting the presence of ostomy effluent. The sensing accessory 12 may be configured to communicate leakage detection signals to the wearable subsystem 20.

The wearable subsystem 20 may be a wearable device powered by an internal battery (not shown). The wearable device 20 may be configured to perform at least some processing of leakage detection signals and to alert a user of a leakage event. The wearable subsystem 20 may be configured to electronically communicate through a wire or wireless communication system with the mobile application. Such electronic communications may include raw data as acquired from the sensing accessory 12 or a leak status for all or part of the sensing accessory 12. The wearable device 20 may also communicate system conditions, such as the presence of a sensing accessory, a faulty sensor, or battery state. The mobile application may be a digital subsystem installed on the mobile device 22. The mobile application may be configured to further process leak detection data and provide an alert or other information about an ostomy appliance to a user.

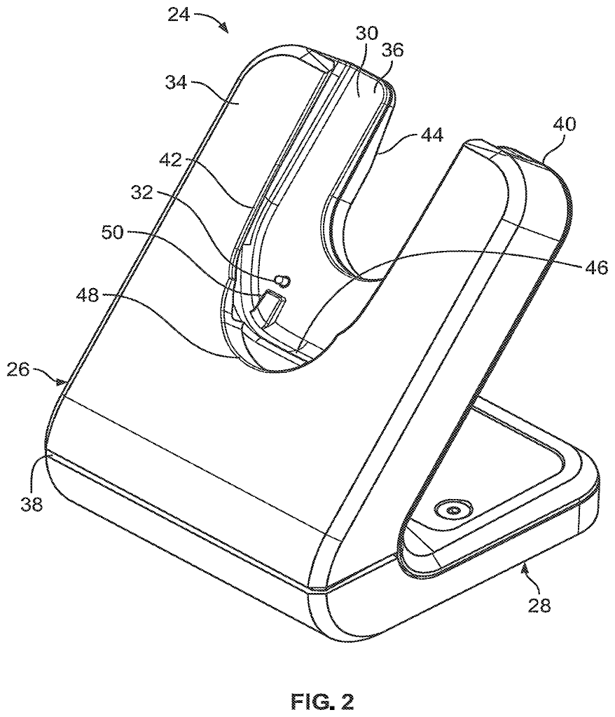

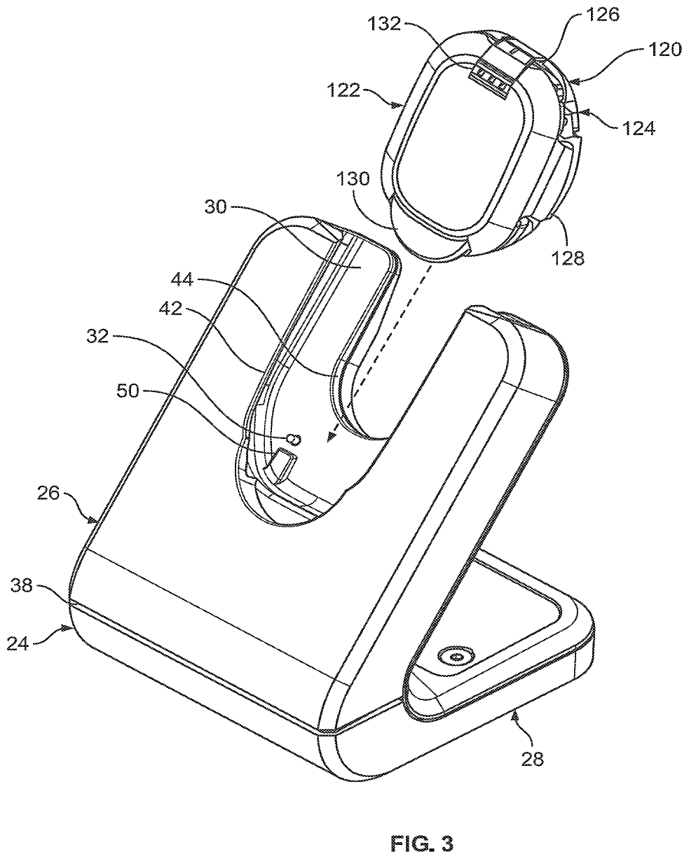

Referring now to the figures, FIGS. 2-13 show a charging dock 24 according to example embodiments. The charging dock 24 may generally include a top housing 26, a bottom housing 28, a slide-in mechanism 30, and a charging pin 32. The slide-in mechanism 30 may include a cavity configured to receive the wearable device 20. (FIGS. 3-5). For example, the dimensions of the cavity are similar to the dimensions of the wearable device 20 so that the wearable device can fit within the cavity of the slide-in mechanism 30. The wearable device 20 may engage and/or connect to the charging pins 32 within the slide-in mechanism 30 for charging.

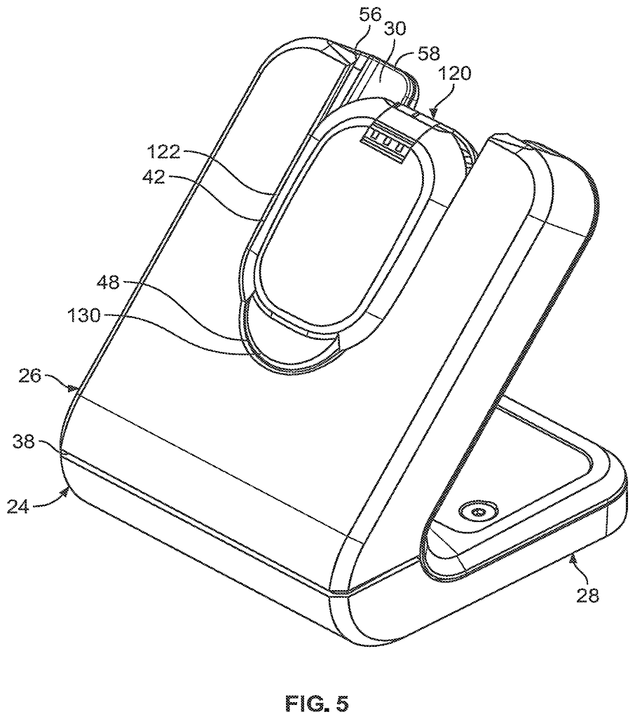

FIG. 2 illustrates a perspective view of the charging dock 24. The charging dock 24 may further include a first wall 34, a second wall 36, a connection section 38, a top end 40, a first opening 42, a second opening 44, a cavity end 46, a lid cavity 48 and a ramp member 50. The first and second wall 34, 36 may define the slide-in mechanism 30. The first opening 42 may be located on the first wall 34. The second opening 44 may be located on the second wall 36. The connection section 38 may be located between the top housing 26 and bottom housing 28. The top end 40 may be located opposite of the bottom housing 28 on the top housing 26. The cavity end 46 may be located within the slide-in mechanism 30 and configured to abut the wearable device 20 when docked. The lid cavity 48 may be located on the first wall 34 near the cavity end 46 and be configured to receive a lid lip 130 (FIG. 5).

The ramp member 50 may be located near the cavity end 46 and aligned with the charging pins 32. The charging pins 32 may be spring pins. The ramp member 50 may be located directly next to the charging pin 32. In other embodiments, the ramp member 50 may be located near the cavity end positioned in the middle of the cavity.

FIG. 3 illustrates a front perspective view of the charging dock 24 and a wearable device 120 aligned for docking. The wearable device 120 may include a top device housing 122, bottom device housing 124, a hinge 126, a latching mechanism 128, a lid lip 130 and one or more indicator lights 132. The wearable device 120 may slide into the slide-in mechanism 30 between the first and second wall 34, 36 through the top end 40.

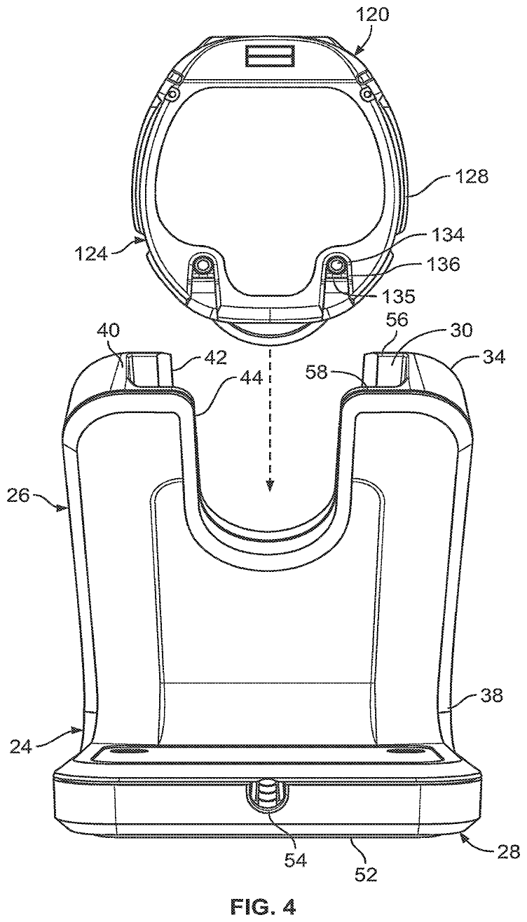

FIG. 4 illustrates a back view of the charging dock 24 and the wearable device 120 aligned for docking. The charging dock 24 may further include an electrical outlet 54, a first peripheral edge 56, and a second peripheral edge 58. The electrical outlet 54 may be located on the bottom housing and configured to connect to an electrical plug for supplying electrical power. The first peripheral edge 56 can be located on the first wall 34. The second peripheral edge 58 can be located on the second wall 36. The wearable device 120 may include charging pads 134, protrusion members 135, and aligning openings 136. The charging pads 134 can electrically connect to the pins 32 for charging. The protrusion members 135 include a peak-like shape or ramp for interacting with the charging pins 32 and ramp member 50.

The slide-in mechanism 30 can align and secure a tight fit of the wearable device 20 within the charging dock 24. The ramp member 50 can stop the wearable device 20 from rotating while inserted into the charging dock 24. Such engagement can ensure that the wearable device 20 is unable to shift out of the position to enable charging of the device, so that no matter how the user inserts the device into the charging dock 24, the charging pins 32 will make continuous contact with the charging pads 134.

The charging dock 24 can comprise a snap positioning mechanism for tactile/auditory notification of correct placement of the wearable device 120 within the charging dock 24. The snap positioning mechanism can be cooperatively provided by both the charging dock 24 and the wearable device 120. When the wearable device 120 is pushed or inserted into the charging dock 24, the spring-loaded pins 32 can engage the protrusion members 135 and become compressed until the user provides sufficient force to move device 120 over the peak of the protrusions. The protrusion members 135, after the peak shape or ramp, can slope or ramp downward and can allow the spring-loaded pins 32 to contact the charging pads 134. The charging dock 24 housing can prevent any further forward movement of the wearable device 120 to ensure that device 120 is in the correct charging location. This snap-action combined with the hard stop against the charger housing can create tactile and auditory feedback to a user that the wearable device 120 is positioned correctly in the charging dock.

FIG. 5 illustrates a front perspective view of the charging dock 24 and the wearable device 120 fully inserted in the charging position. As shown schematically in FIG. 5, the wearable device 120 is within the slide-in mechanism 30. When inserted within slide-in mechanism 30, the wearable device 120 can be viewable through the first opening 42. According to example embodiments shown in FIG. 5, the latching mechanism 128 can be covered by the first and second peripheral edges 56, 58. The latching mechanism 128 can be covered so that the wearable device 120 may not be accessed during charging. The sensing accessory 12 may be detached from the wearable device 120 and the contacts within the wearable device 120 may be covered and/or concealed while the wearable device is in the charging position for user safety. The lid section 48 can ensure that the wearable device 120 is inserted correctly without a connection to the sensing accessory 12 and the lid lip 130 can make contact with the lid section 48 to ensure that wearable device 120 is fully engaged into the charging dock 24.

A user may simply slide the wearable device 120 into the cavity of the slide-in mechanism 30 for docking. A user may then use the first and second openings 42, 44 to grasp onto the wearable device 120 and undock the wearable device 120 by sliding it out. Such configuration can allow for easier access to, and gripping on, the wearable device 120 with one hand. The bottom housing 28 of charging dock 24 may act as a base and support top housing 26 in an upright pitched position relative base and an underlying support surface to allow for a large portion of the top housing 26 to be exposed while wearable device 120 is in the charging position. More particularly, the top housing 25 may be angled back and slope over at least a portion of the bottom portion 28 so that the first opening 42 in first wall 34 can face outward. The first opening 42 may allow the one or more lights 132 to be visible which may indicate charging status. The one or more lights 132 can include a charging status indicator.

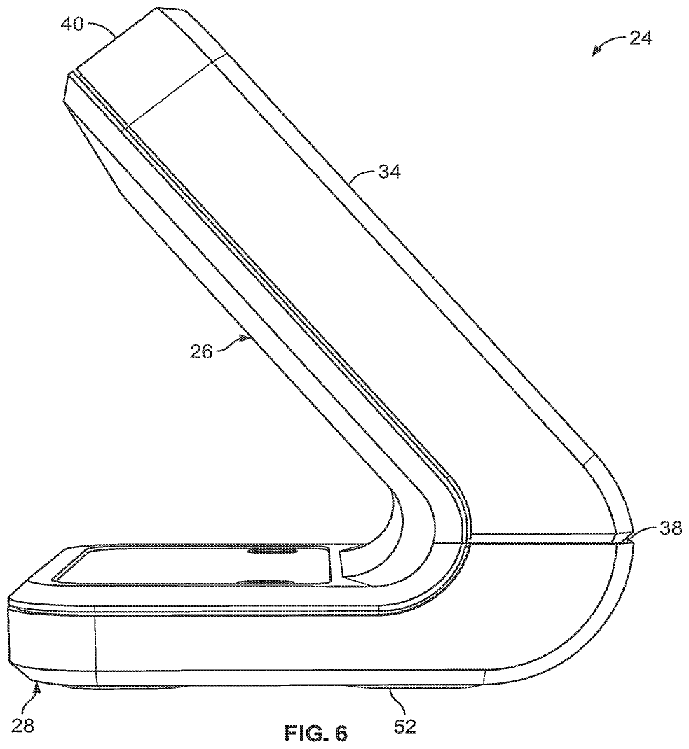

FIG. 6 illustrates a side view of the charging dock 24. The charging dock 24 may include a pad member 52. The pad member 52 may be located on the bottom housing 28 opposite the top housing 26. The pad member 52 may be configured to provide friction between the charging dock 24 and an underlying support surface to prevent charging dock 24 from sliding. According to example embodiments, the pad member 52 may be made out of an elastomer material.

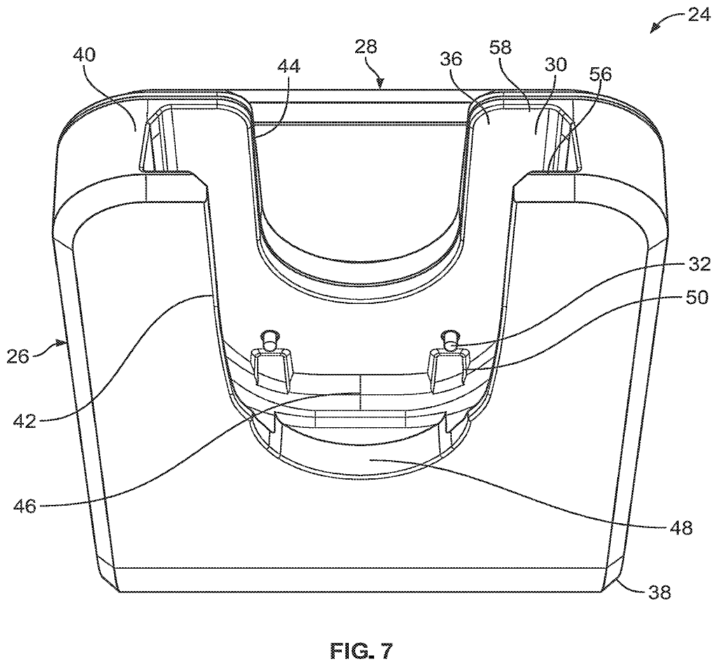

FIG. 7 illustrates a top perspective view of the charging dock 24. As shown schematically in FIG. 7, the slide-in mechanism 30 can have round edges that can match the outer edges of the wearable device 120. The lid cavity 48 can have an opening that can match the lid lip 130.

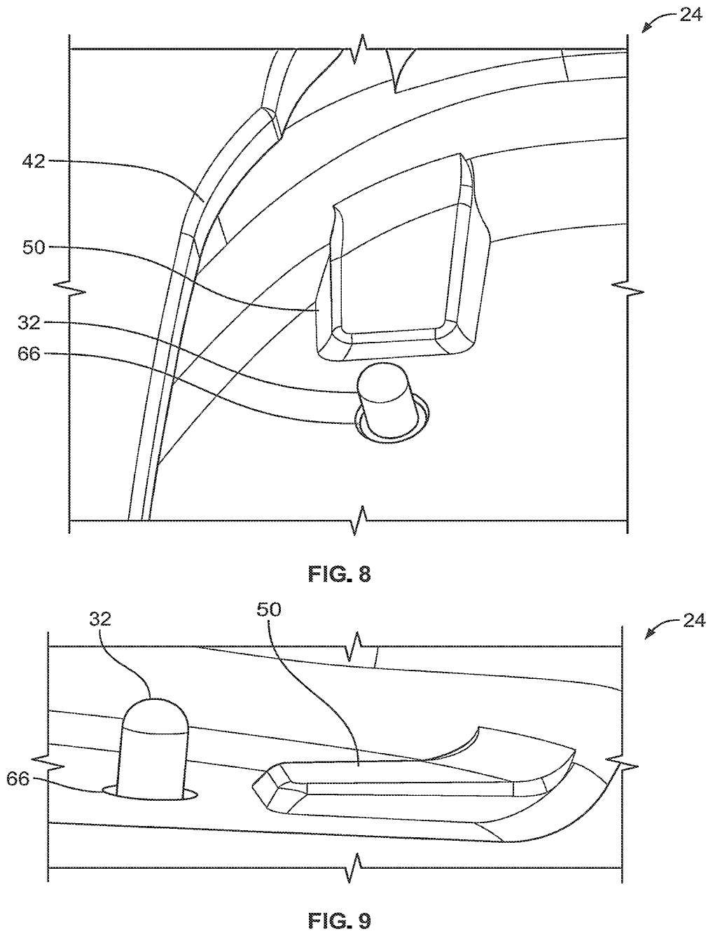

FIG. 8 illustrates a detailed front section view of the charging dock 24. As shown schematically in FIG. 8, the charging dock 24 can include a pin hole 66. According to example embodiments, the pin hole 66 can be located on the second wall 36 and configured to receive charging pin 32. Ramp member 50 can be located and/or aligned adjacent to pin hole 66 and charging pin 32. Ramp member 50 may have sloping and/or round edges to facilitate alignment of the wearable device 120 with the charging dock 24 upon being inserted therein. More particularly, aligning openings 136 on the wearable device 120 (see FIG. 4) can slidably engage with and slide over ramp member 50 to guide wearable device 120 into the charging position within charging dock 24. The aligning openings 136 and the ramp members 50 can secure the charging pins 32 with the charging pads 134 while the wearable device is docked.

FIG. 9 illustrates a side view of the charging dock 24. As shown schematically in FIG. 9, the ramp member 50 can be sloped to provide surface areas to make contact with the aligning opening 136. The charging pin 32 can have a round top for making contact with a surface.

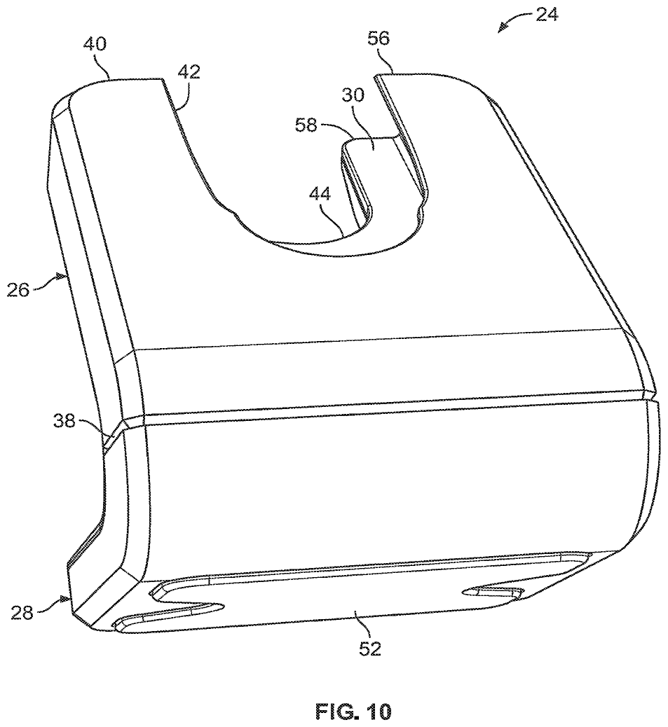

FIG. 10 illustrates a bottom perspective view of the charging dock 24. As shown schematically in FIG. 10, the pad member 52 can have a low profile for limiting the distance of the bottom housing 28 from the support surface. The pad member 52 can have an I-shaped area.

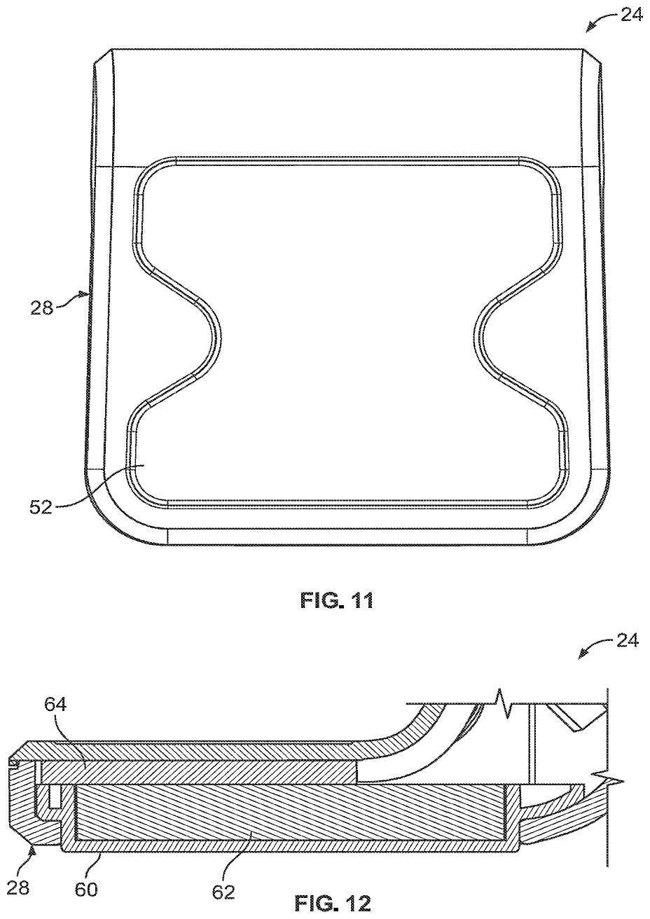

FIG. 11 illustrates a bottom view of the charging dock 24. As shown schematically in FIG. 11, the pad member 52 can have the I-shaped area. In another embodiment, the pad member 52 can have an X-shaped area or similar shape that maximized the amount of area covered on the bottom housing 28.

FIG. 12 illustrates a cross-sectional side view of the charging dock 24. According to example embodiments shown in FIG. 12, charging dock 24 may further include an inner housing 60, a first steel plate 62, and a second steel plate 64. Steel plate 62 may be located within the inner housing 60. The steel plates 62, 64 can provide weight on the bottom housing 28 to create stability for preventing the charging dock 24 from tipping over, and in cooperation with pad member 52, to further prevent dock 24 from sliding on an underlying support surface.

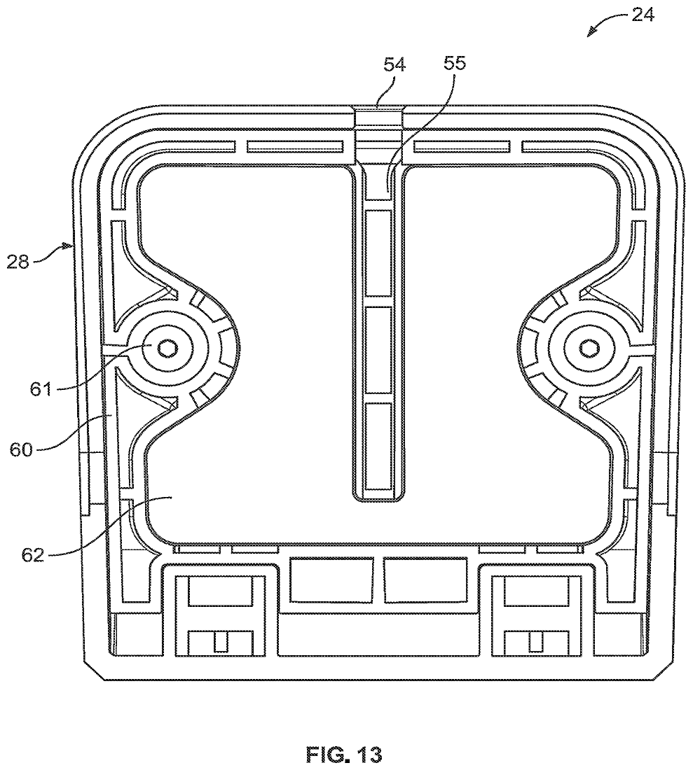

FIG. 13 illustrates a bottom cross-sectional side view of the charging dock 24. The bottom housing 28 can include an electrical pathway 55, and a connecting member 61. The electrical pathway 55 provides a path for electrical wires to be housed and connect the electrical outlet 54 to an electronic board 74. The connecting member 61 can secure the components in the bottom housing 28. As shown schematically in FIG. 13, the steel plate 62 can be shaped to fit within the housing and provide space for other components like connecting member 61 and electrical pathway 55.

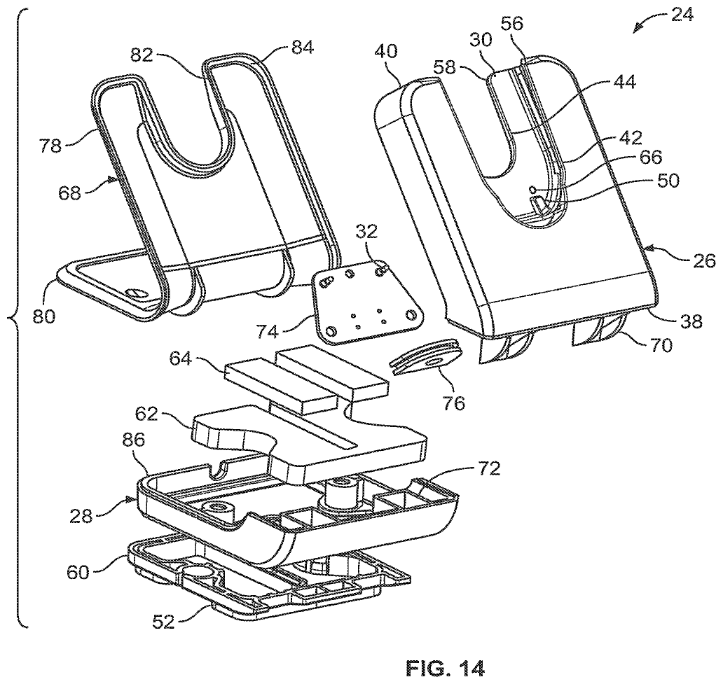

FIG. 14 illustrates an exploded side view of the charging dock 24. According to example embodiments shown in FIG. 14, charging dock 24 can further include a back cover 68, a connect member 70, a receiving connect member 72, an electronic board 74, a mount 76, a top wall cover 78, a bottom wall cover 80, grooves 84, and edges 86.

The back cover 68 can include the top wall cover 78 and the bottom wall cover 80. The top cover wall 78 can cover the back of the top housing 26 and enclose and secure electronic components within the top housing 26. The top wall cover 78 can include a top cover opening 82. The top cover opening 82 can be the same size and shape as the second opening 44. The bottom wall cover 80 can cover the bottom housing 28. The bottom wall cover 80 may include connecting members to secure the back cover 68 to the charging dock 24. The back cover 68 can include grooves 84 for securing the edges 86 of the top and bottom housing 26, 28. The back cover 68 can provide back support to the top housing 26 and can secure the top housing 26 to the bottom housing 28. The connect member 70 can be located on the top housing. The receiving member 72 can be located on the bottom housing 28. The receiving member 72 can be configured to receive the connect member 70 so that the top housing 26 is connected to the bottom housing 28 at the connection section 38.

According to example embodiments, the electronic board 74 can be located within the top housing 26 and configured to hold and connect the charging pins 32 to the electrical outlet 54. The mount 76 can be located underneath the electronic board 74 and configured to align the charging pins 32 to the pin hole 66. The electronic board 74 can further hold electronic components for charging the wearable device.

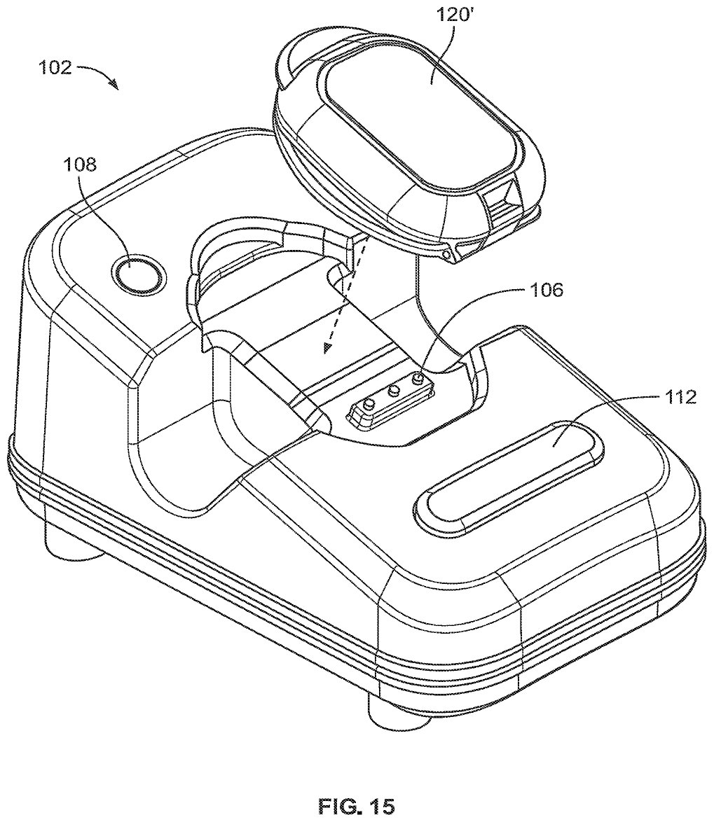

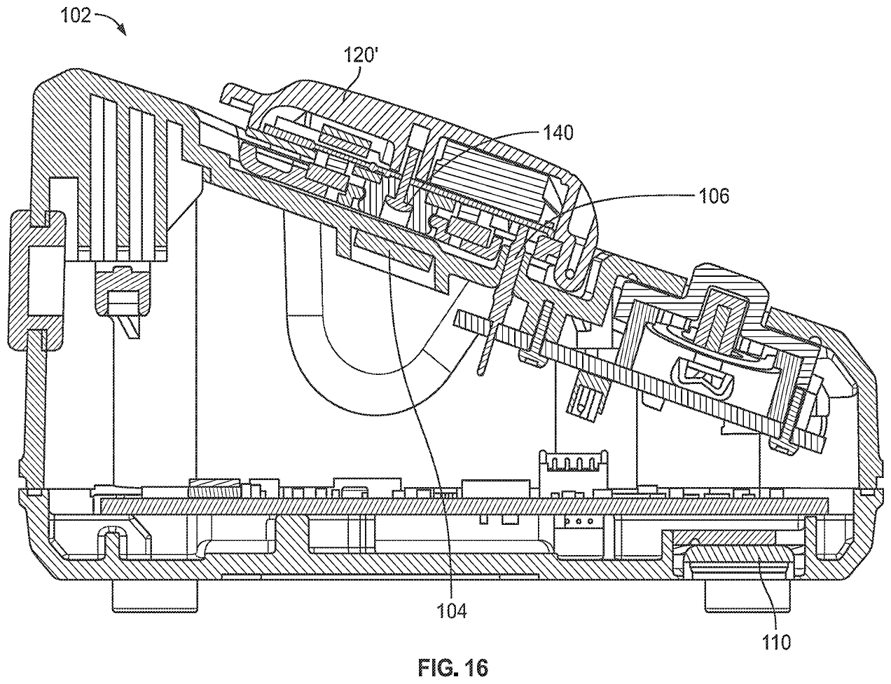

Referring to FIGS. 15 and 16, a charging dock 102 may include a magnetic docking mechanism according to an embodiment. The charging dock 102 may be configured to charge a wearable device 120′ generally similar to the charging dock 24. The charging dock 102 may comprise a magnet 104 and spring contact pins 106. The charging dock 102 may be configured to magnetically pull and secure a wearable device 120′ in a charging position via the magnet 104 and a metal center pin 140 provided in the wearable device 120′. The charging dock 102 may be configured such that when the wearable device 120′ is magnetically pulled into the charging position, the spring contact pins 106 are depressed by the wearable device 120′ and an electrical connection between the charging dock 102 and the wearable device 120′ is established.

In an embodiment, the charging dock 102 may include a status light 108 configured to indicate a status of the wearable device 120′ and/or convey a message to a user. The status light 108 may include various LED colors and/or pulse combinations. In an embodiment, the status light 108 may be configured to indicate a battery level of the wearable device 120′ during charging. The status light 108 may also be configured to alert a leak state to the user. In an embodiment, the charging dock 102 may include a speaker 110 configured for audible alarms. The charging dock may be configured to work in conjunction with an application to configure types of alerts, format of alarm (LED and/or sound), and timeframes for alarms and alerts. The charging dock 102 may be configured so that alarms only operate at night during a timeframe set by a user to alert the user of a leakage during sleep. The status light 108 may be configured to indicate when the charging dock 102 is in a such alert active timeframe. The charging dock 102 may feature a button 112 for silencing the alarms.

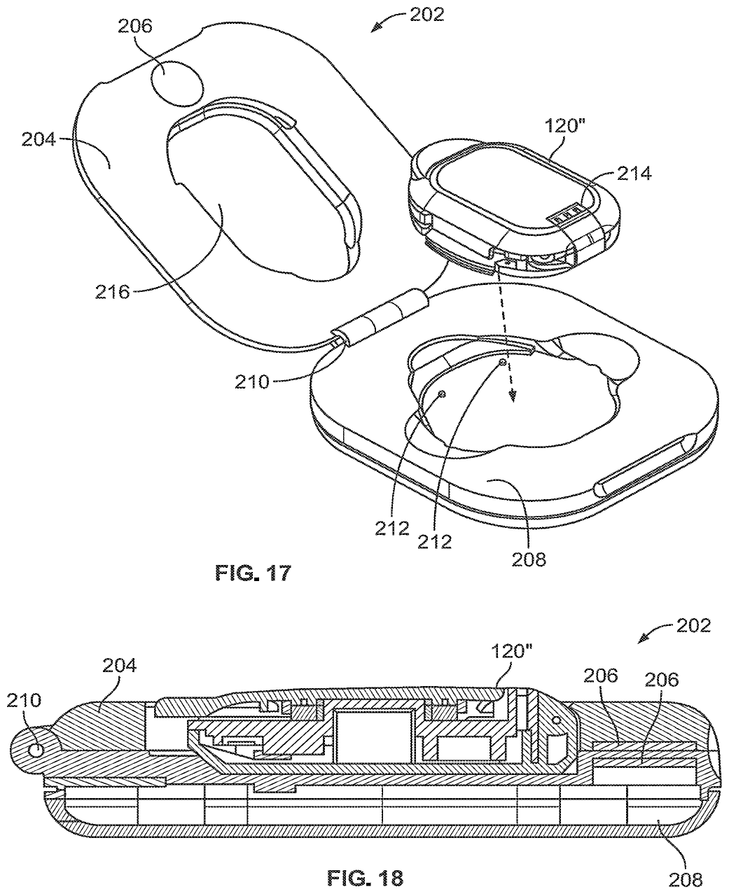

FIGS. 17 and 18 show a charging dock 202 according to another embodiment. The charging dock 202 may be configured to charge a wearable device 120″ generally similar to the charging dock 24, 102 and may generally comprise a hinged top 204 and a base 208 connected by a hinge 210. FIG. 17 is an illustration of the charging dock 202 in an open position, and FIG. 18 is a cross-sectional view of the charging dock 202 with the wearable device 120″ received therein in a closed charging position. The charging dock 202 may include a magnet-magnet or magnet-ferromagnetic metal connection 206 configured to hold the hinged top 204 and the base 208 in a closed position with the wearable device 120″ received therein for charging as shown in FIG. 18.

The charging dock 202 may be configured such that the hinged top 204 may clamp over the wearable device 120″ in a charging position, wherein the wearable device 120″ may be received in the base 208. When the charging dock 202 is closed and in the charging position, the wearable device 120″ may be pressed down to make an electrical connection with the charging dock 202 via spring contact pins 212. The charging dock 202 may be configured such that the spring contact pins 212 align with charging pads the wearable device 120″ when the wearable device 120″ is received in the base 208.

The magnet-magnet or magnet-ferromagnetic metal connection 206 may magnetically attach the hinged top 204 to the base 208 to hold the charging dock 202 in the closed and charging position. The charging dock 202 may be configured such that it does not close when a sensor is connected to the wearable device 120″ and/or does not open while the wearable device 120″ is charging for user safety. The charging dock 202 may be configured such that a top portion of the wearable device 120″ may be visible when the charging dock 202 is closed and in a charging position to allow a user to see status LEDs 214 on the wearable device 120″. The status LEDs 214 may be configured to indicate various status and conditions of the wearable device 120″, for example, a charging status. In the embodiment shown in FIGS. 17 and 18, the hinged top 204 may include an opening 216 configured to allow a portion of the wearable device including the status LEDs 214 to be visible while the charging dock 202 is closed and in a charging position.

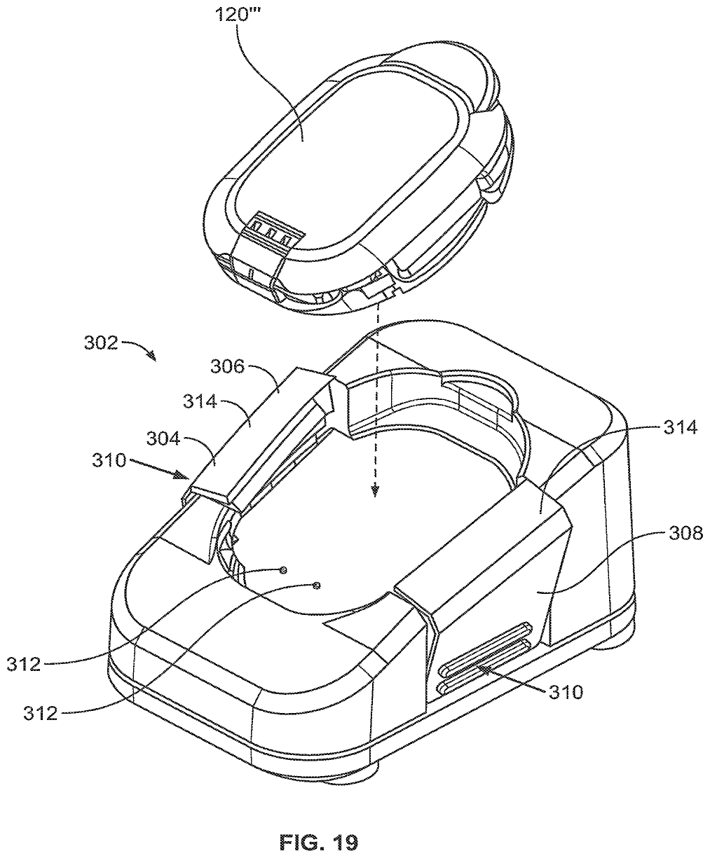

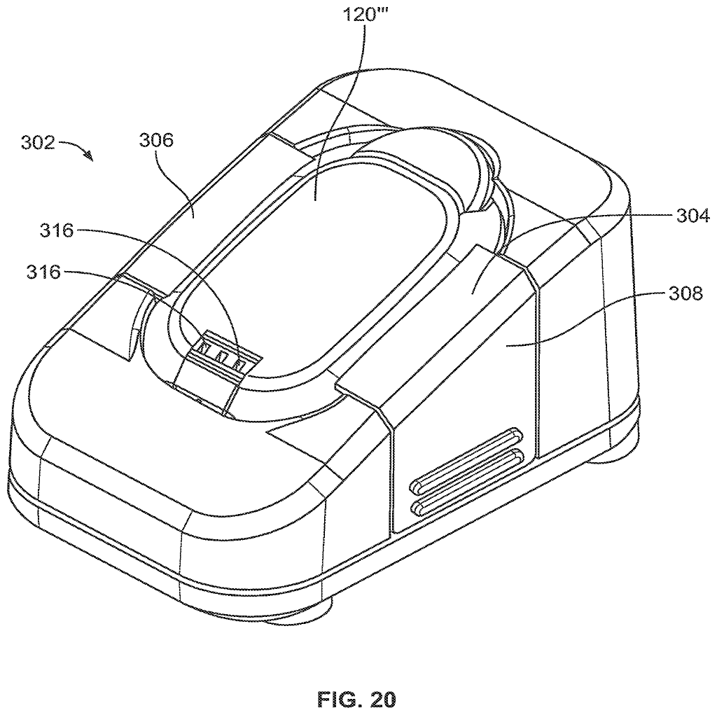

FIGS. 19 and 20 show a charging dock 302 according to yet another embodiment. The charging dock 302 may be configured to charge a wearable device 120″′ generally similar to the charging dock 24, 102, 202 and may include spring contact pins 312. The charging dock 302 may include a latch mechanism 304 for securing a wearable device 120″′ to the charging dock 302, and engaging and compressing charge pads (not shown) provided on the wearable device 120″′ against the spring contact pins 312 in a charging position. The latch mechanism 304 may include two panel latches 306, 308, each attached to a side of the charging dock 302. The latch mechanism 304 may be configured such that when the panel latches 306, 308 are simultaneously squeezed at lower portions 310 as shown in FIG. 19, upper portions 314 of the panel latches 306, 308 hinge outward from the sides of the charging dock 302 to receive the wearable device 120″′. When released, the panel latches 306, 308 latch over the wearable device 120″′ to secure it in place and maintain compression between the charging pads of the wearable device 120″′ and the spring contacts pins 312 of the charging dock 302 in a charging position as shown in FIG. 20. A user may squeeze the lower portions 310 of the panel latches 306, 308 to open the latches to remove the wearable device 120″′.

The charging dock 302 and the wearable device 120″′ may be configured such that a sensor device cannot be connected to the wearable device 120″ and/or the wearable device 120″′ cannot be opened while in the charging position for safety. In the embodiment of FIGS. 19 and 20, at least a portion of the wearable device 120″′ may be exposed and visible in the charging position so that wearable status LEDs 316 are visible to a user to indicate various status of the wearable device 120″′, for example, a charging status.

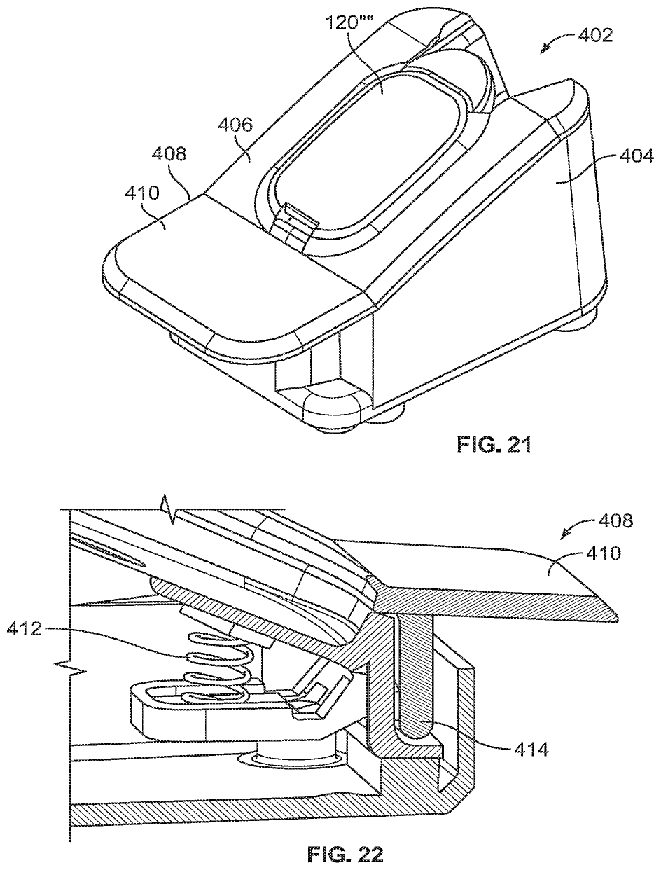

FIGS. 21-24 show a charging dock 402 according to an embodiment. The charging dock 402 may be configured to charge a wearable device 120″″ generally similar to the charging dock 24, 102, 202, 302. The charging dock 402 may generally include a base 404, an upper portion 406, and a press tab release mechanism 408. The base 404 may be configured to receive the wearable device 120″″, and the upper portion 406 may be configured to close over and secure the wearable device 120″″ in a charging position. The press tab release mechanism 408 may include a tab 410 and an internal spring coil 412 connecting the upper portion 406 and the tab 410 at a hinge 414.

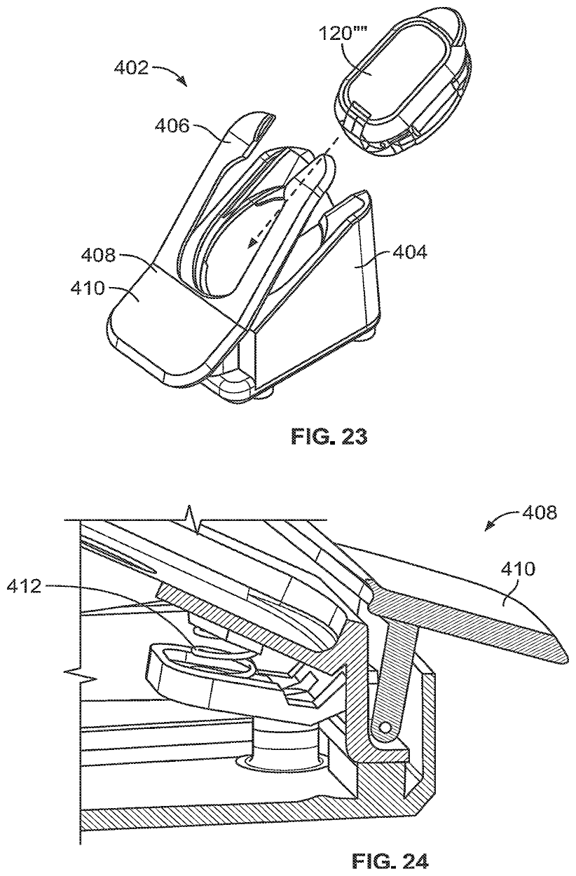

FIG. 21 shows the charging dock 402 closed with the wearable device 120″″ received therein and in a charging position according to an embodiment. The charging dock 402 may be configured such that the upper portion 406 presses the wearable device 120″″ against charging pins provided in the base 404 to establish electrical connection between the wearable device 120″″ and the charging dock 402. As shown in FIG. 23, the charging dock 402 may be configured such that when the tab 410 is pressed down, the internal spring coil 412 is compressed to open the upper portion 406, which allows the wearable device 120″″ to be placed in and removed from the charging dock 402. To close the charging dock 402, the tab 410 may be released so that the internal spring 412 relaxes and the upper portion 406 clamps over the wearable device 120″″ in the charging position. In the embodiments of FIGS. 21-24, the charging dock 402 may be configured such that at least a portion of the wearable device 120″″ may be exposed and visible in the closed charging position to allow status LEDs on the wearable device 120″″ to be visible to a user to indicate various status of the wearable device 120″″, for example, a charging status.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A charging dock comprising:
a housing, wherein the housing comprises a first and second wall;
a slide-in mechanism defined by the first and second wall and configured to receive a wearable device, wherein the first wall comprises a first opening for accessing the wearable device, and wherein the first wall comprises at least one peripheral edge that is configured to extend over the wearable device and prevent the wearable device from opening while docked;
a contact pin located within the slide-in mechanism and configured to charge the wearable device; and
a ramp located within the slide-in mechanism and configured to couple with the wearable device to prevent rotation of the wearable device while docked.

2. The charging dock of claim 1, wherein the contact pin comprises springs.

3. The charging dock of claim 1, wherein the second wall comprises a second opening for accessing the wearable device, and wherein the second wall comprises at least one peripheral edge that extends over the wearable device.

4. The charging dock of claim 1, wherein the housing comprises a top housing and bottom housing, wherein the top housing comprises the first and second wall.

5. The charging dock of claim 4, wherein the bottom housing comprises a base material configured to provide friction.

6. The charging dock of claim 5, wherein the base material comprises an elastomer.

7. The charging dock of claim 4, wherein the bottom housing comprises a weight plate configured to provide stability and prevent the charging dock from sliding on a flat surface.

8. The charging dock of claim 1, wherein the first opening allows for a light on the wearable device to be visible, wherein the light comprises a charging status indicator.

9. The charging dock of claim 1, wherein the first and second openings are configured to allow the wearable device to be un-docked with one hand.

10. A charging dock comprising:
a top housing;
a bottom housing connected to the top housing, wherein the bottom housing comprises a weight plate configured to provide stability and prevent the charging dock from sliding on a surface;
a slide-in mechanism defined in the top housing, wherein the slide-in mechanism comprises a contact pin configured to charge a wearable device; and
a base material located on the bottom housing and configured to provide friction between the charging dock and a surface.

11. The charging dock of claim 10, wherein the base material comprises an elastomer.

12. The charging dock of claim 10, wherein the top housing comprises a first and second wall, wherein the first wall comprises a first opening for accessing the wearable device, and wherein the first wall comprises at least one peripheral edge that extends over the wearable device and is configured to prevent the wearable device from opening while docked.

13. The charging dock of claim 12, wherein the second wall comprises a second opening for accessing the wearable device, and wherein the second wall comprises at least one peripheral edge that extends over the wearable device.

14. The charging dock of claim 13, wherein the first and second openings are configured to allow the wearable device to be un-docked with one hand.

15. The charging dock of claim 10, wherein the charging dock further comprises:
a ramp located within the slide-in mechanism and configured to couple with the wearable device to prevent rotation of the wearable device while docked.

16. A method comprising:
receiving a wearable device into a charging dock, wherein the wearable device comprises an aligning opening, a protrusion member, and a charging pad, and wherein the charging dock comprises:
a housing, wherein the housing comprises a first and second wall;
a slide-in mechanism defined by the first and second wall;
a contact pin located within the slide-in mechanism and configured to charge the wearable device, wherein the contact pin comprises a spring; and
a ramp located within the slide-in mechanism;
compressing the contact pin with the protrusion member;
providing tactile and auditory feedback as the contact pin passes the protrusion member and is decompressed;
engaging the contact pin with the charging pad; and
engaging the aligning opening with the ramp to secure the contact pin on the charging pad.

17. The method of claim 16, further comprising:
securing a latching mechanism between the first and second wall, wherein the latching mechanism is located on an outer edge of the wearable device and cannot be opened within the charging dock.

18-21. (canceled)
